(19) United States
(12) Patent Application Publication
(10) Pub. No.: US 2026/0130838 A1
(43) Pub. Date: May 14, 2026

(54) HAIR CARE FORMULATION

(71) Applicants: Dow Silicones Corporation, Midland, MI (US); Rohm and Haas Company, Collegeville, PA (US)

(72) Inventors: Margery Cortes-Clerget, Midland, MI (US); Marc-Andre Courtemanche, Midland, MI (US); Nisaraporn Suthiwangcharoen, Midland, MI (US); Dawn Carsten, Sanford, MI (US)

(21) Appl. No.: 19/121,156

(22) PCT Filed: Oct. 12, 2023
(86) PCT No.: PCT/US2023/076636

§ 371 Date: Apr. 15, 2025

Related U.S. Application Data
(60) Provisional application No. 63/416,951, filed on Oct. 18, 2022.

(51) Int. Cl. A61K 8/73; A61Q 5/00

(52) U.S. Cl. CPC A61K 8/73; A61Q 5/002

(57) ABSTRACT

A hair care formulation is provided, including: a conditioning polymer; wherein the conditioning polymer comprises a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 24; wherein the conditioning polymer has a degree of substitution of —Si(R¹)₃ groups, DS, of 1.7 to 3; and wherein the conditioning polymer is free of vinylic carbon.

The present invention relates to a hair care formulation. In particular, the present invention relates to a hair care formulation containing: a conditioning polymer; wherein the conditioning polymer comprises a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 24; wherein the conditioning polymer has a degree of substitution of —Si(R¹)₃ groups, DS, of 1.7 to 3; and wherein the conditioning polymer is free of vinylic carbon.

Conventional hair conditioners are popular with consumers for treating hair. Silicone based conditioning agents are the most commonly used conditioning agent in hair conditioner formulations. However, there are growing concerns among some consumers regarding the persistence and potential toxicity of certain conventional silicone based conditioning agents or trace compounds incorporated with such conventional silicone based conditioning agents in the environment, particularly for D4 and D5 conditioners. Accordingly, there has been a growing interest in the development of new conditioning agents for use in hair conditioner formulations.

In U.S. Pat. No. 5,879,670, Melby et al disclose a non-silicon containing amphyolyte polymer for use as a conditioning agent for treatment of a keratin-containing substrate. In particular, Melby et al disclose novel conditioning polymer containing (meth)acrylamidopropyltrimethyl ammonium chloride, meth(acrylic acid) or 2-(meth)acrylamido-2-methylpropane sulfonic acid and, optionally, a C₁₋₂₂ alkyl (meth)acrylate and the use thereof in a cosmetically acceptable medium for the treatment of a keratin-containing substrate (preferably, mammalian hair; more preferably, human hair).

Notwithstanding, there is a continuing need for new hair conditioning agents that provide conditioning benefits. There is also a continuing need for new hair conditioning agents having an increased bio carbon content when compared with conventional hair conditioning agents.

The present invention provides a hair care formulation, comprising: a conditioning polymer; wherein the conditioning polymer comprises a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 24; wherein the conditioning polymer has a degree of substitution of —Si(R¹)₃ groups, DS, of 1.7 to 3; and wherein the conditioning polymer is free of vinylic carbon.

The present invention also provides a method of conditioning hair, comprising: selecting a hair care formulation of the present invention; wetting a plurality of strands of mammalian hair with water; and applying the hair care formulation to the wetted plurality of strands of hair to provide a plurality of strands of treated hair.

### DETAILED DESCRIPTION

We have surprisingly found an effective conditioning polymer, wherein the conditioning polymer is a functionalized maltodextrin comprising a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 24; wherein the functionalized maltodextrin has a degree of substitution of —Si(R¹)₃ groups, DS, of 1.7 to 3; wherein the functionalized maltodextrin is free of vinylic carbon; wherein the conditioning polymer is a biobased and biodegradable material; and wherein conditioning polymer imparts a frizz control benefit to treated hair and restores hydrophobicity to damaged hair.

Unless otherwise indicated, ratios, percentages, parts, and the like are by weight.

The term “dextrose equivalent, DE” as used herein and in the appended claims refers to the degree of starch hydrolysis, specifically, the reducing value of a starch hydrolysate material compared to the reducing value of an equal weight of dextrose, expressed as percent, dry basis, as measured by the Lane and Eynon method described in Standard Analytical Method E-26, Corn Refiners Association, 6^{th} edition, 1977, E-26, pp. 1-3. For example, a maltodextrin with a DE of 10 would have 10% of the reducing power of dextrose which has a DE of 100.

The term “vinylic carbon” as used herein and in the appended claims refers to a carbon that is involved in a double bond with another carbon.

The term “free of vinylic carbon” as used herein and in the appended claims in reference to the functionalized maltodextrin means that the functionalized maltodextrin contains less than the detectable limit of vinylic carbon.

The term “dermatologically acceptable” as used herein and in the appended claims refers to ingredients that are typically used for topical application to the skin, and is intended to underscore that materials that are toxic when present in the amounts typically found in skin care compositions are not contemplated as part of the present invention.

The term “damaged human hair” as used herein and in the appended claims refers to at least one of chemically damaged human hair (e.g., human hair damaged from chemical treatments such as dyeing, bleaching, perming); thermally damaged human hair (e.g., human hair damaged from exposure to heat via ironing, forced drying, styling); and physically damaged human hair (e.g., human hair damaged from physical abuse such as friction, pulling, curling).

The term “aesthetic characteristics” as used herein and in the appended claims in reference to visual and tactile sensory properties (e.g., smoothness, tack, lubricity, texture, color, clarity, turbidity, uniformity).

Preferably, the hair care formulation of the present invention is selected from the group consisting of a rinse off conditioner formulation and a leave on conditioner formulation. More preferably, the hair care formulation of the present invention is a leave on conditioner formulation.

Preferably, the hair care formulation of the present invention, comprises: a conditioning polymer (preferably, 0.01 to 100 wt % (more preferably, 0.01 to 60 wt %; still more preferably, 0.5 to 25; most preferably, 1 to 5 wt %), based on weight of the hair care formulation, of the functionalized maltodextrin); wherein the conditioning polymer comprises a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7); wherein the conditioning polymer has a degree of substitution of —Si(R¹)₃ groups, DS, of 1.7 to 3 (preferably, 1.8 to 3; more preferably, 2 to 3; still more preferably, 2.1 to 2.8; yet more preferably, 2.1 to 2.65; most preferably, 2.1 to 2.5); and wherein the conditioning polymer is free of vinylic carbon; and optionally, a dermatologically acceptable carrier (preferably, 0 to 99.99 wt % (more preferably, 25 to 99.99 wt %; still more preferably, 50 to 99.5 wt %; most preferably, 80 to 99 wt %), based on weight of the hair care formulation, of the dermatologically acceptable carrier). Preferably, the hair care formulation of the present invention, comprises: a conditioning polymer (preferably, 0.01 to 100 wt % (more preferably, 0.01 to 60 wt %; still more preferably, 0.5 to 25; most preferably, 1 to 5 wt %), based on weight of the hair care formulation, of the functionalized maltodextrin); wherein the conditioning polymer comprises a maltodextrin base polymer functionalized with —Si(R¹)₃ groups; wherein the —Si(R¹)₃ groups are linked to the maltodextrin base polymer through a C—O—Si bond; wherein each R¹ is independently a C₁₋₁₀ linear or branched, saturated alkyl group; wherein the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7); wherein the conditioning polymer has a degree of substitution of —Si(R¹)₃ groups, DS, of 1.7 to 3 (preferably, 1.8 to 3; more preferably, 2 to 3; still more preferably, 2.1 to 2.8; yet more preferably, 2.1 to 2.65; most preferably, 2.1 to 2.5); and wherein the conditioning polymer is free of vinylic carbon; and optionally, a dermatologically acceptable carrier (preferably, 0 to 99.99 wt % (more preferably, 25 to 99.99 wt %; still more preferably, 50 to 99.5 wt %; most preferably, 80 to 99 wt %), based on weight of the hair care formulation, of the dermatologically acceptable carrier).

Preferably, the hair care formulation of the present invention, comprises 0.01 to 100 (preferably, 0.01 to 60 wt %; more preferably, 0.5 to 25; most preferably, 1 to 5 wt %), based on weight of the hair care formulation, of a conditioning polymer; wherein the conditioning polymer comprises a maltodextrin base polymer functionalized with —Si(R¹)₃ groups; wherein each R¹ is independently a C₁₋₁₀ linear or branched, saturated alkyl group (preferably, a methyl group, an ethyl group, a propyl group, a butyl group and a pentyl group; more preferably, a methyl group, an ethyl group, a propyl group and a butyl group; still more preferably, a methyl group, an ethyl group and a propyl group; yet more preferably, a methyl group and an ethyl group; most preferably, a methyl group); wherein the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7); wherein the functionalized maltodextrin has a degree of substitution of —Si(R¹)₃ groups, DS, of 1.7 to 3 (preferably, 1.8 to 3; more preferably, 2 to 3; still more preferably, 2.1 to 2.8; yet more preferably, 2.1 to 2.65; most preferably, 2.1 to 2.5); and wherein the functionalized maltodextrin is free of vinylic carbon. More preferably, the skin care formulation of the present invention, comprises 0.01 to 100 (preferably, 0.01 to 60 wt %; more preferably, 0.5 to 25; most preferably, 1 to 5 wt %), based on weight of the hair care formulation, of a conditioning polymer; wherein the conditioning polymer comprises a maltodextrin base polymer functionalized with —Si(R¹)₃ groups; wherein the —Si(R¹)₃ groups are linked to the maltodextrin base polymer through a C—O—Si bond; wherein each R¹ is independently a C₁₋₁₀ linear or branched, saturated alkyl group (preferably, a methyl group, an ethyl group, a propyl group, a butyl group and a pentyl group; more preferably, a methyl group, an ethyl group, a propyl group and a butyl group; still more preferably, a methyl group, an ethyl group and a propyl group; yet more preferably, a methyl group and an ethyl group; most preferably, a methyl group); wherein the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7); wherein the conditioning polymer has a degree of substitution of —Si(R¹)³ groups, DS, of 1.7 to 3 (preferably, 1.8 to 3; more preferably, 2 to 3; still more preferably, 2.1 to 2.8; yet more preferably, 2.1 to 2.65; most preferably, 2.1 to 2.5); and wherein the conditioning polymer is free of vinylic carbon.

Preferably, the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7). More preferably, the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7); wherein the maltodextrin base polymer is a straight or branched chain maltodextrin polymer comprising a plurality of glucose structural units. Most preferably, the maltodextrin base polymer has a dextrose equivalent, DE, of 1 to 24 (preferably, 1 to 20; more preferably, 1 to 15; still more preferably, 1 to 10; yet more preferably, 3 to 10; most preferably, 4 to 7); wherein the maltodextrin base polymer is a straight or branched chain maltodextrin polymer comprising a plurality of glucose structural units; wherein 90 to 100 mol % (preferably, 92 to 100 mol %; more preferably, 93 to 100 mol %; most preferably, 94.5 to 100 mol %) of the glucose structural units are connected by α-1,4 linkages and 0 to 10 mol % (preferably, 0 to 8 mol %; more preferably, 0 to 7 mol %; most preferably, 0 to 5.5 mol %) of the glucose structural units are connected by α-1,6 linkages.

Preferably, the maltodextrin base polymer contains less than 0.01 wt %, based on weight of the maltodextrin base polymer, of alternan. More preferably, the maltodextrin base polymer contains less than 0.001 wt %, based on weight of the maltodextrin base polymer, of alternan. Most preferably, the maltodextrin base polymer contains less than the detectable limit of alternan.

Preferably, <0.1 mol % (preferably, <0.01 mol %; more preferably, <0.001 mol %; most preferably, <detectable limit) of the glucose structural units in the maltodextrin base polymer are connected by β-1,4 linkages.

Preferably, <0.1 mol % (preferably, <0.01 mol %; more preferably, <0.001 mol %; most preferably, <detectable limit) of the glucose structural units in the maltodextrin base polymer are connected by β-1,3 linkages.

Preferably, the hair care formulation of the present invention, comprises 0 to 99.99 wt % (more preferably, 25 to 99.99 wt %; still more preferably, 50 to 99.5 wt %; most preferably, 80 to 99 wt %), based on weight of the hair care formulation, of the dermatologically acceptable carrier. More preferably, the hair care formulation of the present invention, comprises 0 to 99.99 wt % (more preferably, 25 to 99.99 wt %; still more preferably, 50 to 99.5 wt %; most preferably, 80 to 99 wt %), based on weight of the hair care formulation, of the dermatologically acceptable carrier; wherein the dermatologically acceptable carrier is selected from the group consisting of water; glycols (e.g., ethylene glycol, propylene glycol, butylene glycol, pentylene glycol, hexylene glycol, dipropylene glycol, ethoxydiglycol); C₁₋₁₀ straight or branched chain alcohols (e.g., methyl alcohol, ethyl alcohol, propyl alcohol, isopropyl alcohol, butyl alcohol, 2-butoxyethanol); ketones (e.g., acetone); acetates (e.g., methyl acetate); butyl cellusolve; dimethicones; polydimethylsiloxanes; alkanes (e.g., isododecane, isohexane); alkanoates (e.g., methyl undecanoate), dermatologically acceptable hydrophobic ester oils (e.g., caprylic capric triglycerides); dicaprylyl carbonate; alkyl benzoates (e.g., C₁₂₋₁₅ alkyl benzoate); hemisqualane; dioctylether; keto acids (e.g., levulinic acid) and mixtures thereof. Still more preferably, the hair care formulation of the present invention, comprises 0 to 99.99 wt % (more preferably, 25 to 99.99 wt %; still more preferably, 50 to 99.5 wt %; most preferably, 80 to 99 wt %), based on weight of the hair care formulation, of the dermatologically acceptable carrier; wherein the dermatologically acceptable carrier is selected to be capable of evaporating upon application of the hair care formulation to the hair (preferably, human hair). Most preferably, the hair care formulation of the present invention, comprises 0 to 99.99 wt % (more preferably, 25 to 99.99 wt %; still more preferably, 50 to 99.5 wt %; most preferably, 80 to 99 wt %), based on weight of the hair care formulation, of the dermatologically acceptable carrier; wherein the dennatologically acceptable carrier includes isododecane; and wherein the dermatologically acceptable organic carrier is selected to be capable of evaporating upon application of the hair care formulation to the hair (preferably, human hair).

Preferably, the hair care formulation of the present invention, optionally, further comprises at least one additional ingredient selected from the group consisting of an antimicrobial agent/preservative (e.g., benzoic acid, sorbic acid, phenoxyethanol, methylisothiazolinone, ethylhexyl glycerin); a rheology modifier (e.g., PEG-150 pentaerythrityl tetrastearate); pH adjusting agent; an antioxidant (e.g., butylated hydroxytoluene); a humectant (e.g., glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, cationic polymeric polyols); a wax; a foaming agent; an emulsifying agent; a colorant; a fragrance; a chelating agent (e.g., tetrasodium ethylene diamine tetraacetic acid); a bleaching agent; a lubricating agent; a sensory modifier; a sunscreen additive; a vitamin; a protein/amino acid; a plant extract; a natural ingredient; a bioactive agent; an anti-aging agent; a pigment; an acid; a penetrant; an anti-static agent; an anti-frizz agent; an antidandruff agent; a hair waving/straightening agent; a hair styling agent; an absorbent; a conditioning agent (e.g., guar hydroxypropyltrimonium chloride, PQ-10, PQ-7); a slip agent; an opacifier; a pearlizing agent and a salt.

Preferably, the hair care formulation of the present invention further comprises a thickener. More preferably, the hair care formulation of the present invention further comprises a thickener, wherein the thickener is selected to increase the viscosity of the hair care formulation, preferably without substantially modifying the other properties of the hair care formulation. Preferably, the hair care formulation of the present invention further comprises a thickener, wherein the thickener is selected to increase the viscosity of the hair care formulation, preferably without substantially modifying the other properties of the hair care formulation and wherein the thickener accounts for 0 to 5.0 wt % (preferably, 0.1 to 5.0 wt %; more preferably, 0.2 to 2.5 wt %; most preferably, 0.5 to 2.0 wt %), based on weight of the hair care formulation.

Preferably, the hair care formulation of the present invention further comprises an antimicrobial agent/preservative. More preferably, the hair care formulation of the present invention further comprises an antimicrobial/preservative, wherein the antimicrobial/preservative is selected from the group consisting of phenoxyethanol, ethylhexyl glycerin, benzoic acid, benzyl alcohol, sodium benzoate, DMDM hydantoin, 2-ethylhexyl glyceryl ether, isothiazolinone (e.g., methylchloroisothiazolinone, methylisothiazolinone) and mixtures thereof. Most preferably, the hair care formulation of the present invention, further comprises an antimicrobial/preservative, wherein the antimicrobial/preservative is a mixture selected from the group consisting of (a) phenoxyethanol and ethylhexyl glycerin and (b) phenoxyethanol and an isothiazolinone (more preferably, wherein the antimicrobial/preservative is a mixture selected from the group consisting of (a) phenoxyethanol and ethylhexyl glycerin and (b) phenoxyethanol and methylisothiazolinone; most preferably, wherein the antimicrobial/preservative is a mixture of phenoxyethanol and ethylhexyl glycerin).

Preferably, the hair care formulation of the present invention optionally further comprises a pH adjusting agent. More preferably, the hair care formulation of the present invention, further comprises a pH adjusting agent, wherein the hair care formulation has a pH of 4 to 9 (preferably, 4.25 to 8; more preferably, 4.5 to 7; most preferably, 4.75 to 6).

Preferably, the pH adjusting agent is selected from the group consisting of at least one of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of at least one of citric acid, lactic acid, sodium hydroxide, potassium hydroxide, triethanolamine, amino-2-methyl-1-propanol. Still more preferably, the pH adjusting agent includes citric acid. Most preferably, the pH adjusting agent is citric acid.

Preferably, the hair care formulation of the present invention contains <0.01 wt % (preferably, <0.001 wt %; more preferably, <0.0001 wt %; most preferably, <detectable limit), based on weight of the hair care formulation, of octamethylcyclotetrasiloxane (D4) decamethylcyclopentasiloxane (D5) and dodecamethylcyclohexasiloxane (D6) combined.

Preferably, the hair care formulation of the present invention contains <0.01 wt % (preferably, <0.001 wt %; more preferably, <0.0001 wt %; most preferably, <detectable limit), based on weight of the hair care formulation, of conditioning silicones (e.g., polydimethylsiloxanes, dimethicone).

Preferably, the hair care formulation is selected from the group consisting of a leave on conditioner or rinse off conditioner; wherein the hair care formulation contains <0.1 wt % (preferably, <0.001 wt %; more preferably, <0.0001 wt %; most preferably, <detectable limit), based on weight of the hair care formulation, of a hair care cleaning surfactant. More preferably, the hair care formulation is selected from the group consisting of a leave on conditioner or rinse off conditioner; wherein the hair care formulation contains <0.1 wt % (preferably, <0.001 wt %; more preferably, <0.0001 wt %; most preferably, <detectable limit), based on weight of the hair care formulation, of a hair care cleaning surfactant; wherein the hair cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof.

Preferably, the method of conditioning hair (preferably, mammalian hair; more preferably, human hair), comprising: selecting a hair care formulation of the present invention; wetting a plurality of strands of hair with water; and applying the hair care formulation to the wetted plurality of strands of hair to provide a plurality of strands of treated hair (preferably, wherein the plurality of strands of hair comprise strands of damaged hair) (preferably wherein the hair care formulation imparts a frizz control benefit to the plurality of strands of treated mammalian hair such that upon drying there are fewer fly away strands of hair in the plurality of strands of treated mammalian hair compared to the same plurality of strands of mammalian hair treated with a formulation minus the conditioning polymer)(preferably, wherein the hair care formulation imparts an improved alignment benefit such that the plurality of strands of treated mammalian hair have a higher alignment coefficient than hair treated with a formulation minus the conditioning polymer measured using RUMBA-Bossa Nova after six passes) (preferably, wherein the hair care formulation imparts restored hydrophobicity to the stands of damaged mammalian hair).

Some embodiments of the present invention will now be described in detail in the following Examples.

### Synthesis S1: Silylated Maltodextrin

Ammonium chloride (412.4 mg, 0.05 eq) and Glucidex® 1 maltodextrin (DE 1 from Roquette) (25.0 g, 0.154 mol, 1.0 eq) were combined in a 2 CV Helicone mixer (CIT) with a stirring speed at 5 Hz. The resulting mixture was transferred to a reactor. Hexamethyldisilazane (80.87 g, 3.25 eq) was then added to the reactor contents dropwise. Dimethyl sulfoxide (10.8 g) was then added to the reactor contents. The reactor was then sealed and continuously flushed with nitrogen. The reactor contents were stirred at 20 Hz. Heat was applied to the reactor using a heating mantle set to a temperature of 40° C. and the stirring was increased to 50 Hz. After 30 min, the heating mantle was set to 50° C. The temperature setting for the heating mantle was then increased to 80° C. over the course of 1 h by 10° C. increments. The reactor contents were stirred for 1 hr after the temperature of the reactor contents reached 71° C. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜46.3 g). The degree of substitution, DS, of the —Si(CH₃)₃ on the maltodextrin base polymer was determined by ¹H NMR to be 1.67.

### Synthesis S2: Silylated Maltodextrin

Ammonium chloride (454.4 mg, 0.05 eq) and Glucidex® 1 maltodextrin (DE 1 from Roquette) (27.0 g, 0.166 mol, 1.0 eq) were combined in a 2 CV Helicone mixer (CIT) with a stirring speed at 5 Hz. The resulting mixture was transferred to a reactor. Hexamethyldisilazane (87.34 g, 3.25 eq) was then added to the reactor contents dropwise. Dimethyl sulfoxide (11.66 g) was then added to the reactor contents. The reactor was then sealed and continuously flushed with nitrogen. The reactor contents were stirred at 20 Hz. Heat was applied to the reactor using a heating mantle set to a temperature of 92° C. and the stirring was increased to 50 Hz. The reactor contents were stirred for 1.5 hr after the temperature of the reactor contents reached 83° C. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜56.6 g). The degree of substitution, DS, of the —Si(CH₃)₃ on the maltodextrin base polymer was determined by ¹H NMR to be 2.1.

### Synthesis S3: Silylated Maltodextrin

Ammonium chloride (445.4 mg, 0.05 eq) and Glucidex® 1 maltodextrin (DE 1 from Roquette) (27.0 g, 0.167 mol, 1.0 eq) were combined in a 2 CV Helicone mixer (CIT) with a stirring speed at 5 Hz. The resulting mixture was transferred to a reactor. Hexamethyldisilazane (60.47 g, 2.25 eq) was then added to the reactor contents dropwise. Dimethyl sulfoxide (11.7 g) was then added to the reactor contents. The reactor was then sealed and continuously flushed with nitrogen. The reactor contents were stirred at 20 Hz. Heat was applied to the reactor using a heating mantle set to a temperature of 55° C. and the stirring was increased to 50 Hz. After 5 min, the heating mantle was set to 102° C. The reactor contents were stirred for 2 hr. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜407.3 g). The degree of substitution, DS, of the —Si(CH₃)₃ on the maltodextrin base polymer was determined by ¹H NMR to be 2.55.

### Synthesis S4: Silylated Maltodextrin

To a 25 mL scintillation vial was added ammonium chloride (33.0 mg, 0.05 eq) and Maltrin M250 maltodextrin (DE 23-27 from Grain Processing Corporation) (2.0 g, 12.3 mM, 1.0 eq). Hexamethyldisilazane (4.48 g, 2.25 eq) was then added dropwise to the vial contents. Dimethyl sulfoxide (1 g) was then added to the vial contents, and the vial was capped with a screw cap septum topped with two vent needles. The vial was placed on an aluminum heating block set at 85° C. for 1.5 hrs. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜˜4.15 g). The degree of substitution, DS, of the —Si(CH₃)₃ on the maltodextrin base polymer was determined by ¹H NMR to be 2.2.

### Synthesis S5: Silylated Cellulose

Polysaccharide (BioFloc XV, 15.0 g, from Tartas) was weighed in a 2 L three-neck flask equipped with a nitrogen inlet and a temperature controller. Solvent (N,N dimethylacetamide, 331 g, from Sigma-Aldrich) was added and the reaction mixture was placed under an atmosphere of nitrogen with an outlet to avoid over-pressurization of the reactor. Silane (hexamethyldisilazane, 30 g, from The Dow Chemical Company) was added at once to the reaction mixture. The mixture was slowly heated to a set temperature of 130° C. and stirred for 7.5 h. The solution was cooled naturally, then xylenes (600 g, from Sigma-Aldrich) was added to the reaction mixture along with additional hexamethyldisilazane (20 g) and the mixture was stirred for 4 h with a set temperature of 125° C. The reactor contents were left to cool down to room temperature overnight. The reactor contents were then transferred to a separatory funnel and subjected to non-solvent precipitation by dropwise addition into 2 L of vigorously stirring methanol. The product was isolated by filtration, and dried in a vacuum oven at 50° C. overnight. The product was then suspended in 500 ml of methanol, then filtered and dried in a vacuum oven at 50° C. overnight. The product was analyzed by attenuated total reflection infrared to determine DS at 2.23.

### Synthesis S6: Silylated Cellulose

Polysaccharide (E-60, 15.2 g, from GP Cellulose) was weighed in a 2 L three-neck flask equipped with a nitrogen inlet and a temperature controller. Solvent (N,N dimethylacetamide, 324 g) was added and the reaction mixture was placed under an atmosphere of nitrogen with an outlet to avoid over-pressurization of the reactor. Silane (hexamethyldisilazane, 50.2 g, from The Dow Chemical Company) was added at once to the reaction mixture, along with the saccharin catalyst (850 mg, from Sigma-Aldrich). The mixture was slowly heated to a set temperature of 130° C. and stirred for 5 h. The solution was cooled naturally, then xylenes (400 g) were added to the reaction mixture and the mixture was stirred at 120° C. for 8 hours. The reactor contents were left to cool down to room temperature overnight. The cooled reactor contents were then transferred to a separatory funnel and subjected to non-solvent precipitation by dropwise addition into 2 L of vigorously stirring methanol. The product was isolated by filtration and dried in a vacuum oven at 50° C. overnight. The product was then suspended in 500 ml of methanol, then filtered again, and dried in a vacuum oven at 50° C. overnight. was analyzed by attenuated total reflection infrared to determine DS at 2.6.

### Synthesis S7: Silylated Maltodextrin

To a 25 mL scintillation vial was added ammonium chloride (33.0 mg, 0.05 eq) and Maltrin M200 maltodextrin (DE range 16.5-19.9 from Grain Processing Corporation)(2.0 g, 12.3 mM, 1.0 eq). Hexamethyldisilazane (3.58 g, 1.80 eq) was then added dropwise to the vial contents. Dimethyl sulfoxide (0.75 g) was then added to the vial contents and the vial was outfitted with a septum cap with two vent needles. The vial was placed on a heating block set at 80° C. for 1 hr. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜3.6 g). The degree of substitution, DS, of the —Si(CH₃)₃ on the maltodextrin base polymer was determined by ¹H NMR to be 2.76.

### Synthesis S8: Silylated Maltodextrin

To a 25 mL scintillation vial was added ammonium chloride (33.0 mg, 0.05 eq) and Maltrin M040 (DE 4-7 from Grain Processing Corporation) (2.0 g, 12.3 mM, 1.0 eq). Hexamethyldisilazane (4.48 g, 2.25 eq) was then added dropwise to the vial contents. Dimethyl sulfoxide (1 g) was then added to the vial contents and the vial was outfitted with a septum cap with two vent needles. The vial was placed on a heating block set at 85° C. for 1.5 hrs. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜4.15 g). The degree of substitution, DS, of the —Si(CH₃)₃ on the maltodextrin base polymer was determined by ¹H NMR to be 2.5.

### Synthesis S9: Silylated Maltodextrin

Ammonium chloride (445.4 mg, 0.05 eq) and Glucidex® 1 maltodextrin (DE 1 from Roquette) (27.0 g, 0.166 mol, 1.0 eq) were combined in a 2 CV Helicone mixer (CIT) with a stirring speed at 5 Hz. The resulting mixture was transferred to a reactor. Hexamethyldisilazane (87.34 g, 3.25 eq) was then added to the reactor contents dropwise. Dimethyl sulfoxide (11.66 g) was then added to the reactor contents. The reactor was then sealed and continuously flushed with nitrogen. The reactor contents were stirred at 20 Hz. Heat was applied to the reactor using a heating mantle set to a temperature of 50° C. and the stirring was increased to 50 Hz. After 20 min, the heating mantle was set to 100° C. The reactor contents were stirred for 2 hrs. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 mL) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜53 g). The degree of substitution, DS, of the —Si(CH₃)₃ on the maltodextrin base polymer was determined by ¹H NMR to be 2.23.

### Synthesis S10: Silylated Maltodextrin

To a 25 mL scintillation vial was added ammonium chloride (33.0 mg, 0.05 eq) and Glucidex® 1 maltodextrin (DE 1 from Roquette) (2.0 g, 12.3 mM, 1.0 eq). Hexamethyldisilazane (4.48 g, 2.25 eq) was then added dropwise to the vial contents. Dimethyl sulfoxide (1 g) was then added to the vial contents and the vial was outfitted with a septum cap with two vent needles. The vial was placed on a heating block set at 85° C. for 2 hrs. The vial contents were then cooled down to <50° C. and diluted with ethyl acetate (150 mL). The organic layer was transferred to a separatory funnel and washed with distilled water three times (3×50 mL). The organic layer was collected in an Erlenmeyer flask, and dried with sodium sulfate. The organic layer was then concentrated under vacuum to yield a fine white powder (˜3.96 g). The degree of substitution, DS, of the —Si(CH₃)₃ on the maltodextrin base polymer was determined by ¹H NMR to be 2.51.

### Synthesis S11: Silylated Maltodextrin

Ammonium chloride (445.4 mg, 0.05 eq) and Glucidex® 1 maltodextrin (DE 1 from Roquette) (27.0 g, 0.166 mol, 1.0 eq) were combined in a 2 CV Helicone mixer (CIT) with a stirring speed at 5 Hz. The resulting mixture was transferred to a reactor. Hexamethyldisilazane (87.34 g, 3.25 eq) was then added to the reactor contents dropwise. Dimethyl sulfoxide (11.66 g) was then added to the reactor contents. The reactor was then sealed and continuously flushed with nitrogen. The reactor contents were stirred at 20 Hz. Heat was applied to the reactor using a heating mantle set to a temperature of 50° C. and the stirring was increased to 50 Hz. After 20 min, the heating mantle was set to 100° C. The reactor contents were stirred for 2 hrs. The heating mantle was then removed and the stirring was decreased to 25 Hz. When the reactor contents cooled to <50° C., stirring was stopped and 400 mL of ethyl acetate was added to the reactor contents. Stirring was then resumed at 25 Hz for 5 min. Stirring was then stopped and the organic layer was transferred to a collection jar. Ethyl acetate (100 ml) was then added to the reactor contents and stirring was resumed at 25 Hz for 5 min. Stirring was then stopped and the organic layer was transferred to the contents of the collection jar. The contents of the collection jar was transferred to a separatory funnel and washed with distilled water two times (2×250 mL). The organic layer was collected in an Erlenmeyer flask, and dried with sodium sulfate. The organic layer was then concentrated under vacuum to yield a fine white powder (˜56.3 g). The degree of substitution, DS, of the —Si(CH₃)₃ on the maltodextrin base polymer was determined by ¹H NMR to be 2.42.

### Synthesis S12: Silylated Maltodextrin

To a 25 mL scintillation vial was added ammonium chloride (24.7 mg, 0.05 eq) and dried Glucidex® 1 maltodextrin (DE 1 from Roquette) (1.5 g, 3.25 eq). Hexamethyldisilazane (4.85 g, 3.25 eq) was then added dropwise to the vial contents. Dimethyl sulfoxide (0.8 g) was then added to the vial contents and the vial was outfitted with a septum cap with two vent needles. The vial was placed on a heating block set at 90° C. for 4 hrs. The vial contents were then cooled down to <50° C. and diluted with ethyl acetate (200 mL). The organic layer was transferred to a separatory funnel and washed with a 50/50 vol/vol mixture of brine and distilled water three times (3×60 mL). The organic layer was collected in an Erlenmeyer flask, and dried with sodium sulfate. The organic layer was then concentrated under vacuum to yield an off white crystalline solid, that was easily reduced to a fine powder with a spatula. The product powder was dried under vacuum in an oven at 50° C. for 5 hrs. The degree of substitution, DS, of the —Si(CH₃)₃ on the maltodextrin base polymer was determined by ¹H NMR to be 2.42.

### Synthesis S13: Silylated Maltodextrin

To a 25 mL scintillation vial was added ammonium chloride (24.7 mg, 0.05 eq) and dried Maltrin M150 maltodextrin (DE 13-17 from Grain Processing Corporation)(1.5 g, 1.0 eq). Hexamethyldisilazane (4.85 g, 3.25 eq) was then added dropwise to the vial contents. Dimethyl sulfoxide (0.8 g) was then added to the vial contents and the vial was outfitted with a septum cap with two vent needles. The vial was placed on a heating block set at 90° C. for 2.5 hrs. The vial contents were then cooled down to <50° C. and diluted with ethyl acetate (200 mL). The organic layer was transferred to a separatory funnel and washed with a 50/50 vol/vol mixture of brine and distilled water three times (3×60 mL). The organic layer was collected in an Erlenmeyer flask, and dried with sodium sulfate. The organic layer was then concentrated under vacuum to yield an off white crystalline solid, that was easily reduced to a fine powder with a spatula. The product powder was dried under vacuum in an oven at 50° C. for 5 hrs. The degree of substitution, DS, of the —Si(CH₃)₃ on the maltodextrin base polymer was determined by ¹H NMR to be 2.64.

### Synthesis S14: Silylated Maltodextrin

To a 25 mL scintillation vial was added ammonium chloride (33.0 mg, 0.05 eq) and dried Glucidex® 2 maltodextrin (DE 2 from Roquette) (2.0 g, 1.0 eq). Hexamethyldisilazane (4.48 g, 2.25 eq) was then added dropwise to the vial contents. Dimethyl sulfoxide (1.0 g) was then added to the vial contents and the vial was outfitted with a septum cap with two vent needles. The vial was placed on a heating block set at 75° C. for 2 hrs. The vial contents were then cooled down to <50° C. and diluted with ethyl acetate (100 mL). The organic layer was transferred to a separatory funnel and washed with a 50/50 vol/vol mixture of brine and distilled water three times (3×60 mL). The organic layer was collected in an Erlenmeyer flask, and dried with sodium sulfate. The organic layer was then concentrated under vacuum to yield an off white crystalline solid, that was easily reduced to a fine powder with a spatula. The product powder was dried under vacuum in an oven at 50° C. for 5 hrs. The degree of substitution, DS, of the —Si(CH₃)₃ on the maltodextrin base polymer was determined by ¹H NMR to be 2.47.

### Solubility Screening (2 wt %)

The solubility of the products of Syntheses S1-S6 and a commercial maltodextrin with a DE of 1 (Glucidex© 1 from Roquette) were assessed in different carriers by combining individually in separate vials the products of Syntheses S1-S6 (0.1 g) and the commercial maltodextrin with various solvents (4.9 g) as noted in TABLE 1. The resulting 2 wt % solutions were stirred with a magnetic stir bar for 1 hour at ˜22° C. The carriers and solubility observations are provided in TABLE 1.

TABLE 1

| | Observation | | | | | | |
|---|---|---|---|---|---|---|---|
| | Solute (2 wt %) in carrier | | | | | | |
| Carrier | S1 | S2 | S3 | S4 | S5 | S6 | Malt⁹ |
| Water | I | I | I | I | I | I | S |
| Ethanol | I | S | S | S | I | I | I |
| Dimethicone, 2 cSt¹ | — | S | S | S | I | S* | I |
| Isododecane | I | S | S | S | I | I | I |
| Isohexadecane | — | S | S | — | — | — | — |
| Methyl undecanoate | — | S | S | S | — | — | — |
| Triglycerides² | — | S | S | S | — | — | — |
| Dicaprylyl carbonate³ | — | S | S | S | — | — | — |
| C₁₂₋₁₅ alkyl benzoate⁴ | — | S | S | S | I | I | I |
| C₁₃₋₁₅ Alkane⁵ | — | S | S | S | — | — | — |
| Dioctylether | — | S | S | S | — | — | — |
| Ethyl PG-Acetal | — | S | S | — | — | — | — |
| Levulinate⁶ | | | | | | | |
| Homosalates⁷ | — | S | S | S | — | — | — |
| Sunflower oil⁸ | — | I | I | S* | I | I | I |

¹DOWSIL ™ 200 fluid (2 cSt) from The Dow Chemical Company
²CRODAMOL ™ GTCC from Croda
³CETIOL ® CC from BASF
⁴CRODAMOL ™ ABLQ from Croda
⁵NEOSSANCE ® hemisqualane from Centerchem
⁶ECOSMOOTH ™ universal fluid 1100 from The Dow Chemical Company
⁷PARSOL ® HMS from DSM
⁸Sunflower oil, USDA Certified Organic from MakingCosmetics
⁹Glucidex ® 1 maltodextran with DE 1 from Roquette.
S Soluble and clear
S* Soluble and slightly hazy
I Insoluble

### Solubility Screening (50 wt %)

The solubility of the products of Syntheses S2-S3 and S7 (2 g) were assessed in isododecane (2 g) as noted in TABLE 2. The resulting 50 wt % solutions were stirred with a magnetic stir bar for 1 hour at ˜22° C. The carriers and solubility observations are provided in TABLE 2.

TABLE 2

| | Observation | | |
|---|---|---|---|
| | Solute (50 wt %) in carrier | | |
| Carrier | S2 | S3 | S7 |
| Isododecane | S | S | S |
| Ethanol | not tested | S | S |

S Soluble and clear

### Viscosity in Isododecane

The product of Synthesis 5-6 and S9 was dissolved in isododecane at different concentrations in as noted in TABLE 3. The viscosity of the resulting solutions were then determined using a Brookfield DV-II I-ultra viscometer, equipped with a SC4-28 spindle at ˜22° C. and 100 rpm. The results are provided in TABLE 3.

TABLE 3

| Solute | Concentration (wt %) | Viscosity (cP) |
|---|---|---|
| Synthesis S5 | 2 | 3,500 |
| Synthesis S6 | 2 | 3,200 |
| Synthesis S6 | 5 | gel |
| Synthesis S9 | 2 | 0 |
| Synthesis S9 | 5 | 2 |
| Synthesis S9 | 20 | 5 |
| Synthesis S9 | 40 | 305 |
| Synthesis S9 | 50 | 1,260 |

### Comparative Example CF1-CF2 and Example F1: Hair Conditioner Formulations

A hair conditioner formulation was prepared in each of Comparative Examples CF1-CF2 and Example F1 having the formulation noted in TABLE 4.

TABLE 4

| | CF1 | CF2 | CF3 | CF4 | CF5 | CF6 | F1 | F2 | F3 |
|---|---|---|---|---|---|---|---|---|---|
| Ingredient | wt % | | | | | | | | |
| Deionized water | 98 | — | — | — | 100 | — | — | — | — |
| Isododecane | — | 98 | 99 | 100 | — | 98 | 98 | 98 | 98 |
| Maltodextrin¹ | 2 | — | — | — | — | — | — | — | — |
| Isododecane (and) | — | 2 | — | — | — | — | — | — | — |
| Dimethiconol² | | | | | | | | | |
| Bis-Diisopropanolamino- | — | — | 1 | — | — | — | — | — | — |
| PG-propyl | | | | | | | | | |
| Dimethicone/Bis-Isobutyl | | | | | | | | | |
| PEG-14 Copolymer (and) | | | | | | | | | |
| Polysorbate 20 (and) | | | | | | | | | |
| Butyloctanol³ | | | | | | | | | |
| Product of Synthesis S6 | — | — | — | — | — | 2 | — | — | — |
| Product of Synthesis S2 | — | — | — | — | — | — | 2 | — | — |
| Product of Synthesis S14 | — | — | — | — | — | — | — | 2 | — |
| Product of Synthesis S8 | — | — | — | — | — | — | — | — | 2 |

¹Glucidex 1 maltodextrin DE 1 from Roquette
²DOWSIL ™ PMX-1505 fluid from The Dow Chemical Company
³DOWSIL ™ CE-8411 smooth plus emulsion from The Dow Chemical Company

### Hair Hydrophobicity

Hair conditioner formulation prepared according to each of Comparative Examples CF1-CF2 and Example F1 were tested on separate 4 g hair tresses (bleached hair round from DeMeo Brothers, Inc.; lot 4506145707). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute.

To measure hydrophobicity of the hair, the tresses were combed straight and then held tightly on both ends with a holder. Ten 30 μL drops of water were placed at different locations on each tress from the root to the tip and observed. The water drops placed on the hair tress treated with the formulation of Comparative Example CF1 were observed to dissipate immediately from the surface of the hair tress. The water drops placed on the hair tress treated with the formulation of Comparative Example CF2 were observed to dissipate from the surface of the hair tress within ˜2 minutes after application. The water drops placed on the hair tress treated with the formulation of Example F1 were observed to remain on the surface of the hair tress for at least 10 minutes after application before dissipating into the hair.

### Drying Time

Hair conditioner formulation prepared according to each of Comparative Examples CF1, CF3-CF4 and Example F1 were tested on separate 4 g hair tresses (bleached hair round tresses from DeMeo Brothers, Inc.; lot 4506145707). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute.

Drying time was evaluated for the treated tresses hung in a controlled atmosphere at room temperature (21° C.) and 50% relative humidity. The tress treated with the formulation of Example F1 was observed to speed up the drying time without creating frizz relative to the other tresses treated with Comparative Examples CF1 and CF3-CF4.

### Frizz Control

Hair conditioner formulation prepared according to each of Comparative Examples CF1, CF3-CF4 and Example F1 were tested on separate 4 g hair tresses (bleached hair round tresses from DeMeo Brothers, Inc.; lot 4506145707). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute.

The treated hair tresses were then hung to dry for 24 hours in a controlled atmosphere at room temperature (21° C.) and 50% relative humidity. The treated hair tresses were then evaluated using a Bolero Lite System from Bossa Nova Vision of Los Angeles Calif. for volume evaluation and frizz analysis. The Bolero Lite System is an imaging system designed to quickly quantify the hair tress volume while also characterizing the fly-away volume of the hair tress (the FAF %). Note that a lower FAF % corresponds to better frizz control performance. The FAF % observed for the treated tresses are reported in TABLE 5.

TABLE 5

| Hair Conditioner Formulation | FAF % |
|---|---|
| Comparative Example CF1 | 27.3 |
| Comparative Example CF3 | 29.4 |
| Comparative Example CF4 | 25.8 |
| Example F1 | 16.6 |

### Static Reduction

Hair conditioner formulation prepared according to each of Comparative Examples CF1, CF3-CF4 and Example F1 were tested on separate 2 g hair tresses (darkly bleached hair tresses from International Hair Importers, Inc.; lot 4507227801). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute.

The treated hair tresses were then hung to dry for 24 hours in a controlled atmosphere at room temperature (21° C.) and 50% relative humidity. The treated hair tresses were then each run between the index and middle fingers very quickly and pictures were immediately taken for comparison. The tresses treated with Comparative Examples CF1 and CF4, respectively, were observed to show the highest static. The tresses treated with Comparative Example CF3 and Example F1, respectively, were observed to exhibit significantly less static. It is hypothesized that the Isododecane (and) Dimethiconol and the product of Synthesis S2 present in Comparative Example CF3 and Example F1 coat the hair strands and insulate them from static charge.

### Fly Away Frizz Control

Hair conditioner formulation prepared according to each of Comparative Examples CF1-CF2, CF5-CF6 and Examples F1-F3 were tested on separate 4 g hair tresses (virgin frizzy hair tresses from International Hair Importers, Inc.; lot 4506169047). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute.

The treated hair tresses were then hung to dry for 24 hours in a controlled atmosphere at room temperature (21° C.) and 50% relative humidity. The treated hair tresses were then evaluated using a Bolero Lite System from Bossa Nova Vision of Los Angeles Calif. for volume evaluation and frizz analysis. The Bolero Lite System is an imaging system designed to quickly quantify the hair tress volume while also characterizing the fly-away volume of the hair tress (the FAF %). Note that a lower FAF % corresponds to better frizz control performance. The FAF % observed for the treated tresses are reported in TABLE 6.

TABLE 6

| Hair Conditioner Formulation | FAF % |
|---|---|
| Comparative Example CF1 | 38.0 |
| Comparative Example CF2 | 38.7 |
| Comparative Example CF5 | 37.8 |
| Comparative Example CF6 | 41.7 |
| Example F1 | 32.4 |
| Example F2 | 34.1 |
| Example F3 | 33.3 |

### Hair Alignment

Hair conditioner formulation prepared according to each of Comparative Examples CF2, CF5-CF6 and Examples F1-F3 were tested on separate 4 g hair tresses (virgin frizzy hair tresses from International Hair Importers, Inc.; lot 4506169047). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute. Hair alignment was measured using RUMBA-Bossa Nova with the alignment coefficient reported for after 0, 2, 4, 6, 8 and 10 passes. The results are provided in TABLE 7.

TABLE 7

| | Alignment Coefficient | | | | | |
|---|---|---|---|---|---|---|
| | 0 | 2 | 4 | 6 | 8 | 10 |
| Formulation | passes | passes | passes | passes | passes | passes |
| Comp. Ex. CF1 | 18.84 | 25.87 | 29.89 | 38.58 | 40.00 | 35.28 |
| Comp. Ex. CF2 | 10.82 | 20.92 | 34.86 | 39.51 | 42.05 | 55.08 |
| Comp. Ex. CF5 | 10.41 | 20.81 | 24.18 | 22.82 | 25.99 | 22.25 |
| Comp. Ex. CF6 | 11.76 | 26.01 | 31.17 | 35.21 | 46.23 | 41.53 |
| Example F1 | 9.78 | 16.27 | 17.44 | 23.93 | 26.07 | 29.96 |
| Example F2 | 14.34 | 22.26 | 27.7 | 35.99 | 30.59 | 35.97 |
| Example F3 | 21.70 | 26.54 | 35.22 | 36.99 | 38.13 | 46.86 |

### Example F4-F5: Hair Conditioner Formulations

A hair conditioner formulation was prepared in each of Examples F4-F5 having the formulation noted in TABLE 8. It was observed that the hair conditioner formulation of Example F4 was hazy while the hair conditioner formulation of Example F5 was clear.

TABLE 8

| | |
|---|---|
| F4 | F5 |

| | |
|---|---|
| Ingredient | wt % |

| | | |
|---|---|---|
| Cyclomethicone and dimethicone¹ | 48.9 | 48.9 |
| Cyclomethicone² | 44.1 | 44.1 |
| Argania spinosa (Argan) kernel oil | 0.4 | 0.4 |
| Ethanol | 4.0 | 4.0 |
| Product of Synthesis S2 | 2.6 | — |
| Product of Synthesis S8 | — | 2.6 |

¹XIAMETER ™ PMX-1411 Fluid from The Dow Chemical Company
²XIAMETER ™ PMX-0245 Fluid from The Dow Chemical Company

### Fly Away Frizz Control

Hair conditioner formulation prepared according to each of Examples F4-F5 were tested on separate 4 g hair tresses (virgin frizzy hair tresses from International Hair Importers, Inc.; lot 4506169047). The hair tresses were first rinsed with water for 30 seconds. Then a 9% w/w aqueous solution of sodium lauryl sulfate (0.2 g/g of hair) was massaged into the hair tresses for 30 seconds. Then the hair tresses were rinsed with water for 60 seconds. The hair tresses were then treated with the hair conditioner at a dosage of 0.15 g/g or hair and massaged onto the hair for 1 minute.

The treated hair tresses were then hung to dry for 24 hours in a controlled atmosphere at room temperature (21° C.) and 50% relative humidity. The treated hair tresses were then evaluated using a Bolero Lite System from Bossa Nova Vision of Los Angeles Calif. for volume evaluation and frizz analysis. The Bolero Lite System is an imaging system designed to quickly quantify the hair tress volume while also characterizing the fly-away volume of the hair tress (the FAF %). Note that a lower FAF % corresponds to better frizz control performance. The FAF % observed for the treated tresses are reported in TABLE 9.

TABLE 9

| Hair Conditioner Formulation | FAF % |
|---|---|
| Example F4 | 44.8 |
| Example F5 | 39.4 |

### Comparative Examples CF7-CF9 and Examples F6-F10: Water Repellency

Water repellency of a film is strongly influenced by surface energy. High water repellency is desirable for skin care applications. The water repellency of a formulation can be evaluated by measuring the water contact angle from a deposited film of the formulation. Specifically, films were coated onto a glass slide (50 m wet thickness) from dispersions formed using the ingredients noted in TABLE 10 using a doctor blade film applicator with the gap set at 6 mils (0.1524 mm) from the as received polymer solutions, and films were air dried in an environmental controlled room (˜22° C. and 50% RH) for at least 72 hours. The water contact angles for the deposited films were then measured (in degrees) at 4 and 120 seconds after water droplets were deposited on the substrate using a drop shape analyzer (Kruss DSA100). The results of the water contact angle measurements are provided in TABLE 10. Higher contact angles indicate greater water repellency. Contact angles of above 90° are considered excellent.

TABLE 10

| | Ingredients | | | Water | |
|---|---|---|---|---|---|
| | | | Iso- | contact | |
| | Polymer | | dodecane | angle (°) | |
| Example | Type | (wt %) | (wt %) | 4 s | 120 s |
| Comp. Ex. CF7 | Silicone Acrylate¹ | 2 | 98 | 104.5 | 102.3 |
| Comp. Ex. CF8 | Modified pullulan² | 2 | 98 | 92.4 | 90.7 |
| Comp. Ex. CF9 | Ethyl cellulose³ | 2 | 98 | 71.3 | 66.3 |
| Example F6 | Synthesis S11 | 2 | 98 | 96.2 | 94.2 |
| Example F7 | Synthesis S12 | 2 | 98 | 108.0 | 93.8 |
| Example F8 | Synthesis S13 | 2 | 98 | 106.1 | 101.5 |
| Example F9 | Synthesis S3 | 2 | 98 | 129.4 | 114.4 |
| Example F10 | Synthesis S3 | 20 | 80 | 112.6 | 100.2 |

¹DOWSIL ™ FA4012 ID acrylates/polytrimethylsiloxy-methacrylate copolymer from The Dow Chemical Company
²TSPL-30-ID isododecane (and) trimethylsiloxysilylcarbamoyl pullan from Shin-Etsu Chemical Co., Ltd.
³ETHOCEL ™ 7 from DuPont

We claim:

1. A hair care formulation, comprising:
a conditioning polymer; wherein the conditioning polymer comprises a maltodextrin base polymer functionalized with —Si(R¹)₃ groups; wherein each R¹ is independently a C₁₋₁₀ linear or branched, saturated alkyl group; wherein the maltodextrin base polymer has a dextrose equivalent, DE, of 1 to 24; wherein the conditioning polymer has a degree of substitution of —Si(R¹)₃ groups, DS, of 1.7 to 3; and wherein the conditioning polymer is free of vinylic carbon.

2. The hair care formulation of claim 1, wherein the maltodextrin base polymer has a dextrose equivalent, DE, of 1 to 20.

3. The hair care formulation of claim 2, further comprising:
a dermatologically acceptable vehicle.

4. The hair care formulation of claim 3, wherein the dermatologically acceptable vehicle is selected from the group consisting of cosmetic oils and dermatologically acceptable organic solvents.

5. The hair care formulation of claim 4, wherein the hair care formulation contains 0.01 to 60 wt % of the conditioning polymer.

6. The hair care formulation of claim 5, wherein each R¹ is a methyl group.

7. The hair care formulation of claim 6, wherein the dermatologically acceptable vehicle is isododecane.

8. A method of conditioning hair, comprising:
selecting a hair care formulation of claim 1;
wetting a plurality of strands of mammalian hair with water; and
applying the hair care formulation to the wetted plurality of strands of hair to provide a plurality of strands of treated hair.

9. The method of claim 8, wherein the hair care formulation plurality of strands of hair comprise strands of damaged hair.

10. The method of claim 9, wherein hydrophobicity is restored to the hair.
